(19) United States
(12) Patent Application Publication
(10) Pub. No.: US 2025/0322939 A1
(43) Pub. Date: Oct. 16, 2025

(54) METHOD AND SYSTEM FOR STREAMLINING MEDICAL OPERATION FLOW

(71) Applicant: OpComm, Inc., St. Louis, MO (US)

(72) Inventors: Raphael SUN, St. Louis, MO (US); Kaushik SRINIVASAN, St. Louis, MO (US); Victor WILCOX, St. Louis, MO (US)

(21) Appl. No.: 18/866,344

(22) PCT Filed: May 17, 2023
(86) PCT No.: PCT/US2023/067143

§ 371 Date: Nov. 15, 2024

Related U.S. Application Data
(60) Provisional application No. 63/342,952, filed on May 17, 2022.

(51) Int. Cl. G16H 40/20; G16H 10/60; G16H 15/00; G16H 20/40; G16H 70/20

(52) U.S. Cl. CPC G16H 40/20; G16H 10/60; G16H 15/00; G16H 20/40; G16H 70/20

(57) ABSTRACT

A method and system for monitoring workflow of different surgical teams performing surgical procedures. The example system provides centralized task lists for each team member or each surgical team. The example system provides communication channels between team members. Finally, the example system allows collection of time data relating to completion of tasks for follow up analysis of workflow. The collected data is used to provide analytical reports to reduce turnover time between surgical procedures.

### PRIORITY CLAIM

The present disclosure claims benefit of and priority to U.S. Provisional Application No. 63/342,952, filed May 17, 2022. The contents of that application are hereby incorporated by reference in their entirety.

### TECHNICAL FIELD

The present invention relates generally to improving medical communications and monitoring medical operation systems, and more specifically to a system that identifies teams of healthcare providers to be involved in a medical procedure such as surgeries, enables communications among said team, monitors communications, and collects and tracks data relating to the same.

### BACKGROUND

Hospitals generally employ specialists for procedures such as surgeries. In the context of a surgery, such specialists often include surgeons, anesthesiologists, scrub nurses, circulating nurses, and other personnel for surgical teams in a surgical center. Additional team members without core duties to the patient may also be interested in a case or added to it. Such team members include charge nurses, operating room (“OR”) operations managers (aka project managers), medical students, interns, residents, and physician assistants of various types.

Surgeries require that each of the team members complete a number of tasks before the surgery may be started. It is desirable that the time required to complete such clinical workflows be minimized to increase the number of surgical procedures that may be performed within a clinic. Further reducing the time between any two surgeries for the efficient operation of the surgical center is desirable as team members may then focus on performing additional surgeries.

Turnover time is defined as the time between surgeries. Currently, approximately 88% of surgeries are delayed. The average duration of such delays is 24 minutes relative to internally established benchmarks of a hospital. An internal benchmark of turnover time for many hospitals may be 45 minutes. There are many causes of excessive turnover time, which include but are not limited to, inefficient scheduling and information flow. Delays impede efficient provision of surgical services.

Thus, there is a need for a system that allows communication between surgical team members to exchange scheduling data to reduce turnover time. There is a need for a system that collects task data to reduce the time required to complete clinical workflows to increase the number of surgical procedures that may be performed within a clinic. There is also a need to reduce the time between any two surgeries for the efficient operation of the surgical center.

### SUMMARY

According to one example, a computer implemented method for coordination of tasks for a team of medical professionals performing a medical procedure is disclosed. Scheduling information for a medical procedure is received. The scheduling information includes team members with computer devices and a set of tasks associated with each of the team members prior to commencing the medical procedure. The set of tasks is provided to each of the team members on the computer devices. A task list interface is generated that includes an input to indicate completion of tasks on the set of tasks associated with each team member. The method includes communicating to the computer devices of all other team members that a task has been completed. The method includes recording when a task has been completed.

Another disclosed example is a system for monitoring medical procedures. The system includes a server storing schedule data of medical procedures. The schedule data includes members of a medical procedure team and task lists for each member of the medical procedure team for a medical procedure. A communications interface communicates with user devices each associated with a member of a team to perform a medical procedure. A controller on each user device is operable to display a task list for each of the members of the medical team based on the schedule data on a display. The controller receives inputs from the medical procedure team member indicating the completion of a task on the task list. The controller transmits data to the server indicating the input of completion of a task on the task list. The controller receives a communication of completion of a task from another medical team member from the server.

Another disclosed example is a system for monitoring medical procedures. The system includes a storage device storing schedule data of medical procedures. The schedule data including members of a medical procedure team and task lists for each member of the medical procedure team for a medical procedure. A communications interface communicates with user devices each associated with a member of a team to perform a medical procedure. A controller is coupled to the storage device and communication interface. The controller sends a task list to the user device associated with each of the members of the medical team based on the schedule data on a display. The controller receives inputs from one of the user devices indicating the completion of a task on the task list. The controller sends a signal for the user devices associated with the team members to update the task lists to indicate the completion of the task.

Another disclosed example is a device for input of completion of tasks for a medical procedure team member. The device includes a display and a communication interface to receive schedule data from an external server. The schedule data includes the members of a medical procedure team and task lists for each member of the medical procedure team for a medical procedure. The device includes a controller that displays a task list for each of the members of the medical team based on schedule data on the display. The controller receives inputs from the medical procedure team member indicating the completion of a task on the task list. The controller transmits data to the external server indicating the input of completion of a task on the task list. The controller receives a communication of completion of a task from another medical team member.

Another disclosed example is a non-transitory, machine readable medium having stored thereon instructions for monitoring preparation for a medical procedure. The stored instructions comprise machine executable code, which when executed by at least one machine processor, causes the machine processor to receive scheduling data for the medical procedure. The scheduling data includes team members with computer devices and a set of tasks associated with each of the team members prior to commencing the medical procedure. The stored instructions cause the machine processor to provide the set of tasks to each of the team members on the computer devices and generate a task list interface that includes an input to indicate completion of tasks on the set of tasks associated with each team member. The stored instructions cause the machine processor to communicate to the computer devices of all other team members that a task has been completed and record when a task has been completed.

The above summary is not intended to represent each embodiment or every aspect of the present disclosure. Rather, the foregoing summary merely provides an example of some of the novel aspects and features set forth herein. The above features and advantages, and other features and advantages of the present disclosure will be readily apparent from the following detailed description of representative embodiments and modes for carrying out the present invention when taken in connection with the accompanying drawings and the appended claims.

### BRIEF DESCRIPTION OF THE DRAWINGS

FIG. 1 is a block diagram of an example surgical workflow and data collection system;

FIG. 2 is a screen image of an initial interface presented to a user of the system in FIG. 1;

FIG. 3A is a screen image of a cases listing interface presented to a user from the initial interface in FIG. 2;

FIG. 3B is a screen image of a case selected from an individualized case listing interface in FIG. 3A with displayed team tasks;

FIG. 3C is a screen image of a case selected from an individualized case listing interface in FIG. 3A with displayed team tasks and different status indicators;

FIG. 3D is a screen image of a team listing selected from the individualized case listing selected from the interface in FIG. 3A;

FIG. 4A is a screen image of a report interface displayed by selecting the reports option of the initial interface in FIG. 2;

FIG. 4B is a screen image of a recurring incidents report generated by selecting a report type from the report interface in FIG. 4A;

FIG. 4C is a screen image of a turnover by specialty report generated by selecting another report type from the report interface in FIG. 4A;

FIG. 4C is a screen image of a task timing report generated by selecting another report type from the report interface in FIG. 4A;

FIG. 4D is a screen image of a turnover by CPT report generated by selecting another report type from the report interface in FIG. 4A;

FIG. 4E is a screen image of a compliance by room report generated by selecting another report type from the report interface in FIG. 4A;

FIG. 5 is a screen image of an interface allowing the management of users of the example system;

FIG. 6A is a screen image of an interface allowing customization of codes for the example system;

FIG. 6B is a screen image of an interface allowing customization of task phases for the example system;

FIG. 6C is a screen image of an interface allowing customization of tasks for the example system; and

FIG. 7 is a flow diagram of an example routine to update task lists for surgical team members and collect data relating to the completing of such tasks.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The example system works with a pool of specialized medical workers and/or licensed individuals, such as the staff of a surgical center within a hospital who are working together to perform a series of tasks such as surgeries. In the example system, a surgical center has pools of surgical staff, including a surgeon, an anesthesiologist, a scrub nurse, and a circulating nurse, that compose a surgical team. This group can further include additional medical personnel, such as surgical specialists, medical students, residents, interns, orderlies, and any other personnel that need to perform perioperative tasks prior to beginning surgery.

The example system provides centralized task lists for each team member. The example system provides communication channels between team members. Finally, the example system allows collection of time data relating to completion of tasks for follow up analysis of workflow.

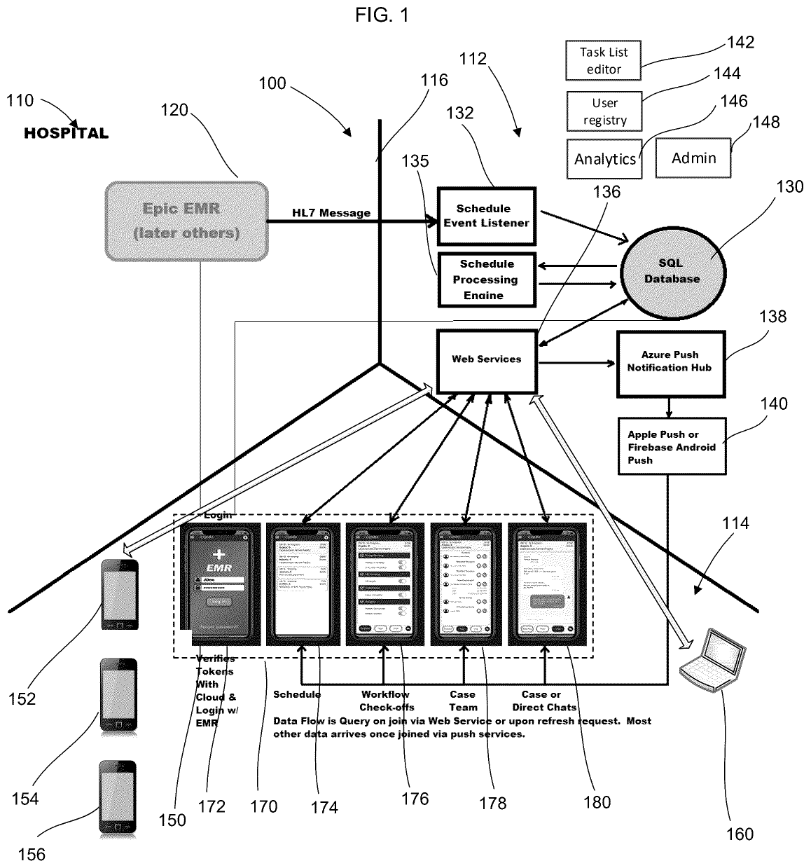

FIG. 1 shows an environment for an example medical procedure tracking system 100. The system 100 includes three subsystems, a local medical database system 110, a Cloud application system 112, and a system of mobile devices 114. The local medical database system 110 includes an electronic medical record (EMR) server 120 that accesses databases that include data relating to patients in a health care facility such as a hospital. Exemplary medical database systems for the EMR server 120 are medical records databases administered by Epic, Cerner, Athena, and Meditech, but other types of medical databases may be used. The EMR server 120 in this example also includes scheduling information for individual patients and optionally also includes different hospital personnel for performing different surgical procedures and associated medical procedure codes (such as the American Medical Association's Current Procedural Technology (CPT) codes) or disease or condition codes (such as the International Classification of Diseases Revision 10 (ICD-10) codes). Although CPT codes are used in this example, other code protocols may be used to identify and classify surgical procedures, patient conditions, and associated tasks.

These subsystems 110, 112, and 114 are all connected to, and configured to communicate with each other over the wide area network 116 such as the cloud or Internet. The connections to the wide area network 116 by the different devices in the subsystems 110, 112, and 114 may be wired or wireless. The EMR server 120 and Cloud applications in the Cloud system 112 may all be implemented on distinct computing devices at separate locations, or any sub-combination of two or more of those entities may be co-implemented on the same computing device.

A wide variety of related data may be accessed the system 100. For example, patient specific data relevant to procedures may be stored in a patient information database that may be operated by the EMR server 120. External databases either in the Cloud or locally may also provide additional data for surgical procedures.

In some implementations, the EMR server 120 manages a database that stores a profile associated with patients. The profile can include, for example, demographic information associated with the user, biometric information associated with the user, medical information associated with the user. The demographic information can include, for example, information indicative of an age of a patient, a gender and/or birth sex of the patient, a race of the patient, a geographic location of the patient, a relationship status, a family medical history, an employment status of the user, including employer provided insurance information, other insurance information for the patient, an educational status of the patient, a socioeconomic status of the patient, or any combination thereof. The medical information can include, for example, including indicative of one or more medical conditions associated with the user, medication usage by the user, or both.

The EMR server 120 may manage electronic medical records, both specific to individual patients and to a larger population of patients. An EMR, sometimes referred to as an electronic health record (EHR), typically contains a medical history of a patient, including previous conditions, treatments, co-morbidities, and current status. The EMR server 120 may be located, for example, at a hospital where any of the user have previously received treatment. The EMR server 120 is configured to transmit EMR data to the Cloud based application in the Cloud system 112. The EMR server 120 may also store emergency procedure case reports (EPCRs) generated by emergency medical service providers.

The cloud application subsystem 112 is a portal that includes an SQL database 130, a schedule event listener 132, a schedule processing engine 134, a web services module 136, a push notification hub 138, and a device push hub 140. In this example, scheduled surgical procedures and related information such as patient data are loaded from the EMR server 120. The EMR server 120 sends schedules for surgeries as well as patient data to the schedule event listener 132 via HL7 messages in this example. Any suitable messaging protocol with appropriate security relating to health care systems instead of the HL7 protocol may be used. The schedule processing engine 134 receives the schedule event data and organizes the schedule according to the preferences of the users. For example, the user may choose to see the cases listed first according to the room number identifier, and then according to the time of day. Alternatively or additionally, the schedule processing engine 134 may arrange the schedule first by surgical pod, and then by a second, and optionally a third schedule parameter such as surgical specialty and the surgeon. Alternatively, the schedule processing engine 134 may sort the case schedule according to individual team members, and a user may view all the cases that they are assigned as well as the general schedule for the medical procedure unit (such as a surgery center).

The schedule processing engine 134 generates and responds to changes in the SQL database 130, which also interacts with the web services module 136 to communicate with individual devices of the relevant team for surgeries. Individual devices may, for example, accept input indicating that a specific user has joined a specific case or a series of cases. The portal of the Cloud system 112 provides all of the functions of the applications that run on the user devices 114 and preferably additionally provides a task list editor 142, an end-user registry 144, an analytics package 146, an administration module 148, or any combination of the foregoing, and most preferably, all of the foregoing.

Medical procedures can be grouped into classes of procedures. A class of procedures may optionally be described by a single CPT code. Similarly, a class of procedures may be described by a group of CPT codes. Other listings for form classes of procedures are by medical specialty, team member, ICD-10 codes, time of day, or whether the procedure is either the first or last procedure of the day. There are other ways to classify procedures, which for brevity are not detailed here.

The task list editor 142 allows an administrator to edit tasks for a single class of procedures, all procedures, or a group of classes of procedures. The individual tasks are assigned or associated with team members associated with the case. In one embodiment, the tasks are preset in the system, but other methods of task determination and assignment may be used.

In some surgical teams the tasks may be broken into four or five groups: (i) tasks for a preoperative nurse, tasks for an OR nurse, tasks for an anesthesiologist, and tasks for a surgeon; or (ii) tasks for registration desk personnel in addition to the four foregoing roles. Example preoperative nurse tasks may include noting when the patient has arrived in the area, confirming that an intravenous line has been started, confirming the pre-operative laboratory testing report is complete and satisfactory, and that the patient has received all required preoperative medicines (such as vaccines). Example OR nurse tasks may include verifying and noting that the OR is ready. Example anesthesiologist tasks may include confirming that the pre-anesthesia exam is complete, confirming the patient has no allergies of concern. Example surgeon tasks may include ensuring that the patient has given proper and adequate consent, the site of surgery has been marked, and the patient has completed an adequate pre-surgical medical history and physical examination. The set of tasks may be standard across different types of medical procedures.

In another embodiment, the task list editor 142 is used to add a task to an individual class of procedures. For example, applicable law may require a special consent for certain types of surgeries. The task list editor 142 can be used to add a second, specific form of consent to the task list but only for procedures that require an extra consent as specified by applicable law. Thus, a standard set of tasks may be provided, and specialized tasks may be added to the sets of tasks. For example, the task lists may be specialized according to an identity of a first physician; an identity of a first and a second physician; a medical procedure identifier code, such as the CPT code; a disease or condition code such as the ICD-10 code; or a location of an operating room.

In another embodiment, a particular surgeon may require a specific set of surgical tools to always be available in the operating room when the particular surgeon is leading the surgery. The task list editor 142 may be used to add a task for a team member other than the surgeon to ensure that the set of surgical tools is in the room and available for use (should a particular surgeon decide that they are needed). However, this task will not show up on cases associated with other surgeons, as they do not require the putative specified tools to be available to them.

The ability to add key steps to the workflow of a team will decrease surgical turnover time by ensuring that standard but specialized steps are presented to the user and team, and recorded when they have been completed, thereby notifying the entire team that they do not need to check that a particular task is completed.

The end-user registry 144 is used for verification of user credentials for users of the system 100. As will be explained, the analytics package 146 provides analysis of collected data on surgical team flow. The analytics package 146 generates reports that may include graphs and tables that are useful to identify bottlenecks, exceptional cases, and other sources of extended turnover time based on analysis of the task data collected by the system 100 from surgical team members. As will be explained, the administration module 148 allows an administrator to adjust configurations such as tasks, task descriptions, and codes for the system 100.

The individual devices 114 include user devices of individuals that are authorized to access the overall system 100. Typically, all potential members of surgical teams may access the system 100. In this example, each team member has a respective user device 150, 152, 154, and 156. Thus, in this example, the user device 150 may belong to a surgeon, the user device 152 may belong to an anesthesiologist, the user device 154 may belong to a scrub nurse, and the user device 156 may belong to a circulating nurse. In this example, the four users are part of a specific surgical team that is scheduled via the EMR server 120 for a surgery case. Other users that may include the users of the user devices 150, 152, 154, and 156 may be members of other surgical teams for other cases. Such additional members may have respective associated user devices. The user devices 150, 152, 154, and 156 may be a mobile device such as a smart phone or any other like device that allows wireless communication. The user devices 114 may also include a computer device 160 that may be a lap top computer, a tablet, a desk top computer, a work station or other computer.

In one embodiment, one or more mobile computing devices may be placed in one or more of the following locations, which may be selected according to frequent work flows (in any combination) of an individual team. Thus, locations may include on the door of an operating room, in the operating room, proximal the OR Board (preferably within 30 feet of the OR Board, more preferably within 12 feet of the OR Board, and independently preferably in a location in which the display surfaces of the OR Board and the mobile computing device are simultaneously visible to a user), in or at the Nurses' Station, near or in the hospital's entry foyer, patient transport center, in the intensive care unit (ICU), in one or more pre-anesthesia rooms, in a patient holding area, in the Registration Area, in the cafeteria, in a break room or sleeping quarters within the hospital, on the medical floor. Mobile computing devices may be used by any user who can log in to the system. One advantage of adding such mobile computing devices in common areas is that users using their personal mobile devices (e.g., cell phones/mobile phones/smart phones) do not need to retrieve the mobile computing device they use most often from a pocket, desk drawer, purse, or other common storage area to access the system. Another advantage of placing one or more mobile computing devices in the device network is that they can easily be mounted to walls and other locations by commercially-available mounting devices without the need to engage in remodeling or to use reconstruction services to medial facilities. That is, nearly no retrofitting is required. Another advantage of placing one or more mobile computing is that such devices are specifically registered with the schedule processing engine and display all the cases to occur in that particular operating room that day. One benefit of this embodiment is that the team has ready access to the application as it is preparing the operating room. In a similar embodiment, one or more mobile computing devices may be placed at other locations within the area in which the procedures are taking place. Preferably, these are placed in areas that are secure from view by non-medical personnel, which helps to ensure the privacy of patients. One of skill in the art will understand that the OR Board is a ubiquitous feature in OR suites that lists information regarding the day's surgical schedule. OR Boards display critical but limited information regarding the day's surgical schedule and are most commonly dry erase boards, but may also be electronic displays which display information from the EMR or another database.

In this example, the mobile user devices 150, 152, 154, and 156 may include virtually any, preferably, mobile computing device that is configured to send and receive information over a wireless capable network, such as the network 116. In this example, the user devices 150, 152, 154, and 156 are web-enabled and may run browser software for the presentation of web pages to the user. Such mobile user devices 150, 152, 154, and 156 may include portable devices such as cellular telephones, smart phones, display pagers, radio frequency (RF) devices, infrared (IR) devices, global positioning devices (GPS), Personal Digital Assistants (PDAs), handheld computers, wearable computers, tablet computers, smart TV's, Streaming Services, Digital Boxes and other integrated devices combining one or more of the preceding devices, and the like. The user devices 150, 152, 154, 156, and 160 may include multiprocessor systems, microprocessor-based, or programmable consumer electronics, and the like. As such, user devices running the application described below may range widely in terms of capabilities and features.

As exampled below, the web-enabled user devices 150, 152, 154, 156, and 160 may include a browser application enabled to receive and to send wireless application protocol messages (WAP), and/or wired application messages, and the like. The user devices 150, 152, 154, and 156, and 160 also include an API that emulates a specific application that may be run in conjunction with the browser application. In one example, the browser application is enabled to employ HyperText Markup Language (HTML), Dynamic HTML, Handheld Device Markup Language (HDML), Wireless Markup Language (WML), WMLScript, JavaScript, Extensible HTML (xHTML), Compact HTML (CHTML), and the like, to display and/or send digital information.

The user devices 150, 152, 154, 156, and 160 may also include at least one client application 170 that is configured to receive control data and/or content from another computing device via a network transmission. The client application 170 may include a capability to provide and receive textual content, graphical content, video content, audio content, and the like. Moreover, the user devices 150, 152, 154, and 156 may be further configured to communicate and/or receive a message, such as through a Short Message Service (SMS), direct messaging (e.g., Twitter), e-mail, Multimedia Message Service (MMS), instant messaging (IM), internet relay chat (IRC), mIRC, Jabber, Enhanced Messaging Service (EMS), text messaging, Smart Messaging, Over the Air (OTA) messaging, or the like, between or with another computing device, and the like.

The network 116 is configured to allow communications between one computing device and another computing device. The network 116 may be enabled to employ any form of computer readable media for communicating information from one electronic device to another. On an interconnected set of LANs, including those based on differing architectures and protocols, a router and/or gateway device acts as a link between LANs, enabling messages to be sent between computing devices. Also, communication links within LANs typically include twisted wire pair or coaxial cable, while communication links between networks may utilize analog telephone lines; full or fractional dedicated digital lines including T1, T2, T3, and T4; Integrated Services Digital Networks (ISDNs); Digital Subscriber Lines (DSLs); wireless links including satellite links; or other communication links known to those of ordinary skill in the art. Furthermore, remote computers and other related electronic devices can be remotely connected to either LANs or WANs via a modem and temporary telephone link.

The network 116 may further include any of a variety of wireless sub-networks that may further overlay stand-alone ad-hoc networks, and the like, to provide an infrastructure-oriented connection. Such sub-networks may include mesh networks, Wireless LAN (WLAN) networks, cellular networks, and the like. The network 108 may also include an autonomous system of terminals, gateways, routers, and the like connected by wireless radio links or wireless transceivers. These connectors may be configured to move freely and randomly and organize themselves arbitrarily, such that the topology of the network 108 may change rapidly and arbitrarily.

The network 116 may further employ a plurality of access technologies including 2nd (2G), 2.5, 3rd (3G), 4th generation (4G), 5th generation (5G) radio access for cellular systems; WLAN; Wireless Router (WR) mesh; and the like. Access technologies such as 2G, 3G, 4G, 5G, and future access networks may enable wide area coverage for mobile devices, such as the user devices 150, 152, 154, and 156 and 160, with various degrees of mobility. For example, the network 116 may enable a radio connection through a radio network access such as Global System for Mobile communication (GSM), General Packet Radio Services (GPRS), Enhanced Data GSM Environment (EDGE), Wideband Code Division Multiple Access (WCDMA), CDMA2000, and the like. The network 116 may also be constructed for use with various other wired and wireless communication protocols, including TCP/IP, UDP, SIP, SMS, RTP, WAP, CDMA, TDMA, EDGE, UMTS, GPRS, GSM, UWB, WiMax, IEEE 802.11x, and the like. In essence, the network 116 may include virtually any wired and/or wireless communication mechanisms by which information may travel between one computing device and another computing device, network, and the like.

In this example, the mobile devices such as the user device 150 include the mobile application 170 of the system 100. Other stand alone devices such as the computer 160 may run a computer version of the application 170 with the same functionality. The mobile application 170 provides a roster of surgeries, a checkable task list, a listing of all team members on a particular surgery, a surgical team-specific chat function that permits one-to-one and one-to-all chat messages, one-click access to phone calls from one end-user on a surgical team to another, and a family chat/family call function. The mobile application 170 generates several sets of interfaces that allows different functions of the system 100 to be accessed by the user. The interfaces generated by the application 170 include a login interface set 172, a schedule display interface set 174, a workflow check-off interface set 176, a case team communication interface set 178, and a patient family communication interface set 180. The data and communications for the interfaces are established through the connection to the network 116 to the Cloud subsystem 112.

The login interface set 172 provides interfaces that ensure that only authorized personnel access the system 100. Any security protocols may be used such as entry of a user name and password. Other protocols such as secondary authentication through another device may also be used. The schedule display interface 174 displays the relevant surgical schedule for each team member. The schedule data is populated from the EMR server 120 through the SQL database 130. Thus, the schedule of procedures may be derived from electronic health records, electronic medical records, or emergency procedure case reports generated by emergency medical service providers. The schedule display interface 174 may provide inputs for users (preferably an administrator) to add additional cases in real-time to a schedule. The additional case may be added with team member assignment, task assignment, OR assignment, and the like.

As will be explained, the workflow check-off interfaces set 176 display interfaces of task lists related to scheduled surgical procedures. Each of the team members on each surgery who are working at a given time have a personal set of tasks that must be completed prior to surgery. When every task for each surgical team member is complete, then the whole surgical team's pre-surgical prerequisite tasks are complete and surgery may begin. The example system 100 provides easy access to a task list for each member of the team. Each of the team members may also view the tasks and status of tasks for other team members via the system 100.

The case team communication interfaces 178 allow a communication channel that is focused on helping the whole surgical team understand the status of any one surgical team member's progress in completing their individual prerequisite tasks. The patient family communication interfaces 180 allows communication to the family or other interested external parties.

The system 100 follows a process flow in coordination with surgical procedure schedules for the hospital. The Cloud subsystem 112 may retrieve the surgery schedule from the EMR server 120, which constitutes an electronic healthcare system of a hospital. Alternatively, the surgery schedule can be entered manually into the portal of Cloud subsystem 112 manually through a computer device such as the workstation computer 160. Alternatively, the schedule stored in the EMR server 120 can be manually updated in the administration module 148 to account for emerging changes in the schedule. First, a surgery schedule is assembled or accessed from the EMR 120. Typically, the surgery schedule will have the patient's name or ID, the type of surgery to be performed, and most commonly will have the specific Operating Room (“OR”) listed as well. The system 100 sends a request to the EMR 12—for some or all of the surgical scheduling information resident within the EMR 120. The system 100 parses the data, organizes it into individual surgical procedures (unless the EMR 120 pushes the data in that format), and displays the surgical information on the user's computing device. The system periodically queries the EMR 120 for changes to the surgical schedule and updates the system, preferably multiple times per minute, optionally once every minute, or 2, 3, 4, 5, 6, 7, 8, 9, or 10 times per hour. If users elect to follow or add themselves to a case, the system 100 maintains that election during and following the update. The surgery schedule is usually dynamic for reasons such as because patients fail to prepare themselves for surgery, fail to show up for surgery, or withdraw consent to surgery, emergencies arise requiring adaptation of the schedule, and surgical procedures may be delayed or run longer than expected which requires schedule-changes. Accordingly, the system 100 combines updates derived from querying the EMR 120 and finding new surgery schedule information, users adding themselves to a case, and manual updates to the surgical system made within the editor function of the system 100.

The system 100 assembles surgical teams for each of the surgeries on the schedules. The surgical teams can be drawn initially or entirely from the EMR server 120, by team members adding themselves to a specific surgery, or by another user adding a team member to a specific surgery. There are multiple ways in which a team member can be added to a case. For example, a potential team member may demonstrate interest in a particular case that is on the surgical schedule by viewing any information about the case. Any information about the case may be viewed by clicking or tapping on that case within the surgical schedule displayed by the system 100. Then, the potential team member's (inferred) interest in the case is interpreted by the system 100 to indicate a desire to join that case. Then, the user who has just viewed the case is added to the team roster of the surgery and any team member viewing the team roster will see that the viewing-user has now joined the surgical team. If that option is disabled in the entire system 100 or if that option is disabled on a particular mobile computing device or computer, then the potential team member may join a case within the application by clicking on the case to be joined. The user may generate an input such as a gesture (e.g., a long-press on the case title) which brings up a menu that includes the options: ‘to join a case’, ‘to follow a case but not join it’, or ‘to leave a case.’ Some or all of these options may be sent to the EMR server 120 and used to assemble surgical teams.

The system 100 presents interfaces on user devices, such as the user devices 150, 152, 154, and 156 that show task lists for each member of a surgical team. Thus, all members of a surgical team and individual members of the surgical team may view a predetermined list of key pre-operative tasks that need to be performed prior to: (a) taking the patient into the operating room and/or (b) formally beginning the surgery. Surgery team members update the system 100 through inputs on an interface such as clicking a slider in the mobile phone application 170 to indicate that a particular task is complete. The system 100 allows a communication medium that is focused on helping the whole surgical team understand the status of any one surgical team member's progress in completing their individual prerequisite tasks.

Not every step that surgical team members will accomplish prior to starting a particular surgery needs to be tracked by the system 100, but critical or key tasks may be monitored. The individual's critical tasks can be customized according to surgery type, personnel assigned or signed into the case, time of day, and other parameters set in the portal on the Cloud system 112. There are options for setting the tasks. The tasks may be set by the system operator, a health care profession, an administrator of a health care facility, of by another end-user.

As surgical team members complete each of the standardized/customized tasks on the personal workflows, they can click a button or slider or icon that indicates that the task is complete. The system 100 collects the time that the task is complete for formulating reports by the analytics package 146. The system also updates the interfaces of task lists displayed to all members of a team to show that the task is complete. The data is collected in a file and can be manually queried or queried by the analytics package 146. The time of completion of a task can be registered according to clock time, elapsed time since the prior surgery has been completed, or elapsed time from the first surgery of the day. For the first surgery of the day, start time can be measured relative to the scheduled start time.

The system 100 is preferably configured with the administration module 148. The administration module 148 allows an administrator to edit task lists. An administrator may access the administration module 148 to make task list modifications. For example, the task list for each type of team member may be edited according to the type of surgery performed. This may be done via the American Medical Association Current Procedural Terminology (CPT) Code. Each surgery type may be identified by one or more CPT codes. Each CPT listed for a surgery on the then-current surgical schedule can have a specific set of standardized tasks. If more than one CPT code is listed, there is the option to select the dominant CPT code and the workflow tasks for only that CPT will be listed. Alternatively, the administration module 148 displays all the tasks for both CPT codes, provided that tasks that are part of more than one CPT code will be displayed by the system 100 only once.

The administrator can configure the system 100 to determine which medical personnel are on a particular team from the surgical schedule, and add or remove specific tasks for the specific surgery or all surgeries of a certain type. Certain medical care organizations may have more than one surgical center and may have different standard-task requirements at each location. The administration module 148 can be configured to account for the location-specific tasks.

The administration module 148 can also manage task lists of all end-users registered to access the system 100 and these lists can contain any combination of personnel name, employee number, telephone number(s), employee or contractor status, roles that person may fill on a surgical team, and the like. The administration module 148 also provides access to the analytics package 146 for generation of analysis reports.

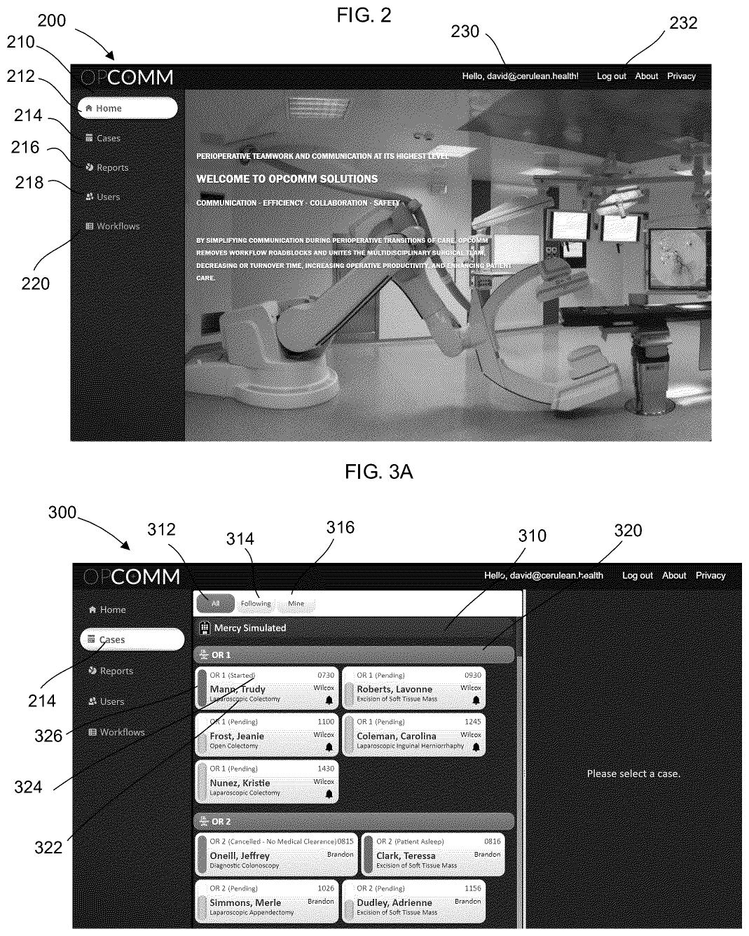

FIG. 2 shows a screen image of an introduction page 200 that is generated by the mobile application 170 on user devices in FIG. 1. The introduction page 200 includes a functional menu 210 that allows a user to select between a home option 212, a cases option 214, a reports option 216, a users option 218, and a workflows option 220. In this example, the introduction page 200 is displayed after a user enters their credentials and logs into the system 100. A user field 230 shows the user identification and a log out option 232 allows a user to log out of the system 100. In this example, the functional menu 210 is displayed on all interfaces generated by the application 170 for navigation of the different functions of the system 100.

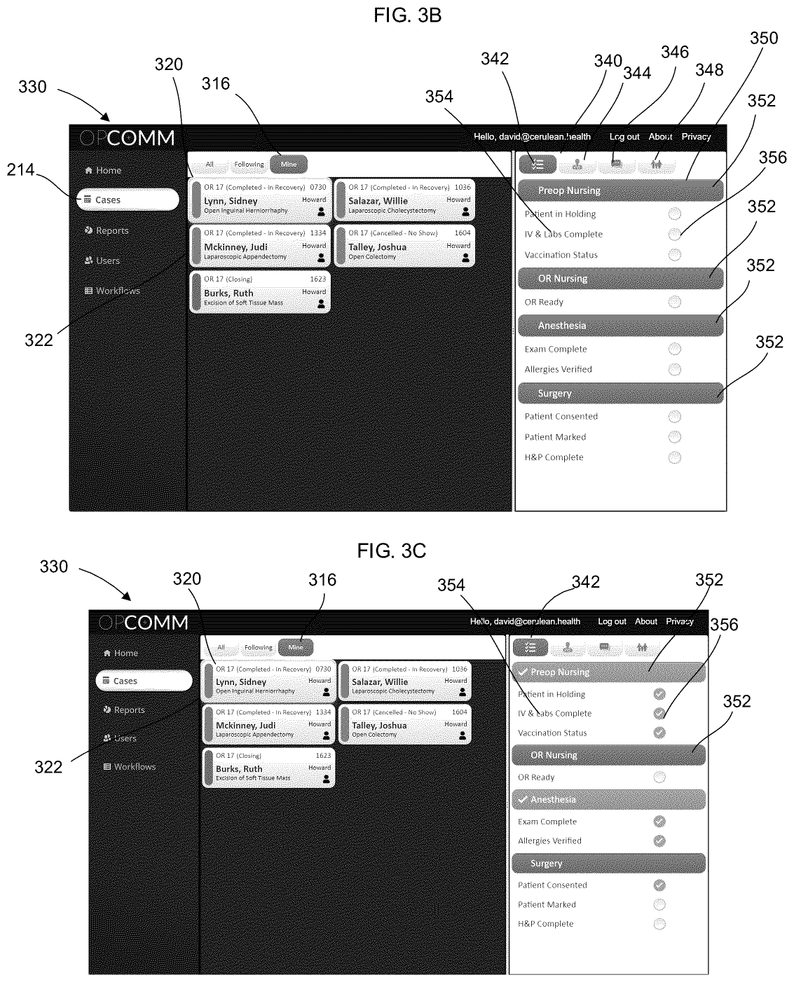

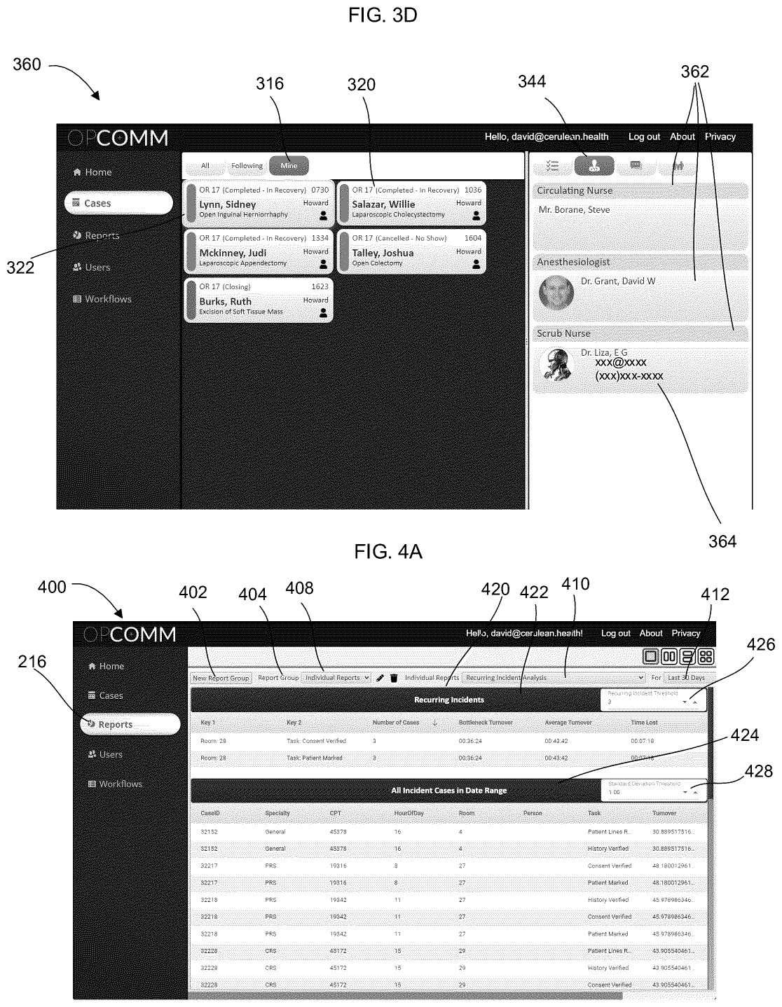

As explained above, the system 100 allows a user to display interfaces that show the status of different surgical procedures (cases). FIG. 3A shows an interface 300 that is displayed when the cases selection 214 is selected from the home page 200. The cases selection 214 causes a case roster window 310 to be displayed for the hospital that is associated with the database system 110. The case roster window 310 includes an “all” option 312, a “following” option 314, and a “mine” option 316. In this example, the “all” option 312 is selected, which shows all cases scheduled for the day. The following option 314 displays an interface that shows all cases being followed by the user. All cases listed or shown in this patent document are simulated-no actual personal healthcare information is displayed and all cases and procedure types are simulated.

The case roster window 310 thus displays each case for each operating room scheduled for the day in a display field 320. In this example, each case has a case tile such as a case tile 322 that is organized by the specific assigned operating room. However, the case roster window 310 can also be organized by surgical specialty (e.g., all orthopedic surgeries in one group, all gastroenterology surgeries in another group, and all neurosurgeries in a third). Cases could also be organized by a key person, such as under each a surgical project manager or anesthesiologist.

Each of the case tiles such as the case tile 322 includes the name of the patient, the type of surgery, and the scheduled time of the surgery. The case tile 322 includes a status field 324 that shows the status such as started, pending, or canceled. A task completion bar 326 shows roughly the number of tasks that have been completed for the case. In this example, the task completion bar 326 may be color coded to indicate the number of tasks completed. For example, when all tasks are completed a dark green bar that fills the entire task completion bar 326 may be displayed, while incomplete tasks lists may be listed in a partial bar of light green. A different color such as red may be displayed in the task completion bar 326 if the case is canceled. Cases that are pending but not started fill the task completion bar 326 with white and if the surgery is complete the task completion bar 326 is filled as gray. Different colors or patterns may be used to indicate the status of the respective case in the case tiles.

The system 100 may allow a multi-case view that permits a project manager, functional coordinator, or other interested medical personnel to review the status of multiple teams on one screen. For example, a multi-case view may show the information relating to up to nine teams on a single screen. In this example, the multi-case view may be displayed on a workstation screen of a workstation such as the computer 160 in FIG. 1.

FIG. 3B shows an interface 330 that is displayed when the “mine” option 316 is selected. Selecting the mine option 316 results in only the cases assigned to the user being displayed in the case roster display window 310. Alternatively, the cases shown in the case roster display window 320 can be toggled to show only the cases to which an end-user is following via the following option 314, or all cases that the end-user is assigned plus cases that that person is following. In the pictured embodiment, the “all”, “following”, and “mine” options are selected by touching or clicking on the button labeled with these descriptions, which in this embodiment is at the top of the case roster. Each of the case tiles 322 in the case roster display window 320 are surgeries assigned to the user in this example. When any of the tiles 322 are selected, a task list popup 340 is displayed. The task list popup 340 has a task list option 342, a persons option 344, a team messaging option 346, and a family chat option 348.

After selecting any particular case in the surgery schedule, the team roster can be selected via the persons option 344. The team roster lists all team members assigned to the selected case. On the team roster, icons for chat and telephone calls can be selected to make a direct connection to between the end-user and any other end-user on the surgical team (for any particular surgery).

Different communications may be set up between team members and team members and individuals associated with patients via the team messaging option 346, direct calling with a hot-link (not shown) on the team roster (FIG. 3D, selectable with persons option 344), and the family chat option 348. Team members assigned or signed into a specific surgery can communicate via a group chat function enabled by the mobile application 170. When a message is available but not read an indicator on both the lock screen of the user device and within interfaces generated by the application that display an indication that there is an unread message. Alternatively, a call to a team member may be enabled by accessing the applicable telephone application on a mobile user device. The system 100 also may use the team roster for a case to enable a group chat (SMS or MMS). In this example, the chat function of the system 100 may use the native text messaging system on the mobile device or may provide an independent chat system that is only accessible within the system 100. The application on the user devices may also enable indicator pop-ups for messages from team members. For example, an indicator pop-up may be a message from the OR nurse to all team members that the operating room and patient are ready, or that the anesthesiologist is rolling the patient from the pre-anesthesia area to the operating room.

Within the case display for any particular surgery, the family chat option 348 selects the ability to open a chat or make a telephone call to the patient's representative (often a family member). There are two options for the family chat: password secured and password-free. The password secure option requires the setup of a password to protect communication with the family. The password-free option allows communication without an extra level of protection. The system 100 can retrieve the family representative's contact information from the EMR server 120 and a call may be enabled or a chat may be enabled by accessing the applicable applications on the user device.

In the example interface in FIG. 3B, the task list option 342 has been selected resulting in a task lists window 350 being displayed. The task lists window 350 shows the tasks for each person on the team separated by team member. Thus, a dynamic task bar 352 is shown for each team member. In this example, four dynamic task bars 352 are shown for each of the four members on the team for the selected case. A list of tasks 354 and corresponding bubbles 356 for that team member are shown under each of the dynamic task bars 352. Once a task is completed, the user may select the appropriate bubble 356 and all devices are updated showing the completion of the task. Each of the dynamic task bars 352 may be color coded. For example, if the dynamic task bar is blue, this may indicate that certain tasks have not been completed, while a green color indicates that all tasks are completed. Support materials may be accessed via an individualized task listing. For example, one task may be obtaining certain consent documents for the surgery. Any of the members of the team can select the consent documents task on their interface, and the document may be display or a list of related documents may be displayed that enable the team member to select a related document and view the related document in the application on the computing device without switching to another application of the computing device

FIG. 3C shows the interface 330 in FIG. 3A when certain tasks are completed and checked off by team members. In this example, the listed tasks in FIG. 3B are still displayed for the selected case tile 322. In this example, the pre-op nursing tasks under the first task bar 352 have been completed. The user has thus entered a check mark on the bubble 356 corresponding to each of the tasks. On inputting the check mark, the user device will send updated task data indicating completion of the task to the Cloud system 112. In this example, there are check mark icons in each of the bubbles 356 associated with the first task bar 352. The task bar 352 may thus be shown with a different color, such as green, to indicate the completion of all tasks by the team member associated with the task bar 352. Other tasks such as those under the last dynamic task bar 352 showing surgeon tasks may be partially completed and thus only one of the bubbles are marked in this example. In this example, the dynamic task bar 352 for the surgeon and the OR nurse is a different color such as blue than the other task bars that may have all tasks completed. The task lists window 350 allows the user to easily view their tasks for each assigned case as well as determine the status of other members of the team. Via the color of the dynamic task bars 352, a user may determine whether the entire team is ready or if individual members are ready for the surgery.

If an end-user selects a case tile, then the team members option 344 displays a screen showing all the persons either assigned to that case, or both assigned to that case and following that case. FIG. 3D shows an interface 360 that is displayed when the team members option 344 is selected from the task list window 350. The team members option 344 will display tiles 362 of all of the persons of the team for the selected case tile 322. Preferably, each person is identified next to a chat icon and a telephone icon enabling the end-user to open a chat or call with any other end-user on a given case. The end-user does not need to be assigned to the particular case to open the call or chat message. Selecting any of the display tiles 362 such as the display tile 364 will highlight the selected tile and display contact information such as phone number and email address for the team member. Other contact information such as a pager number may also be displayed.

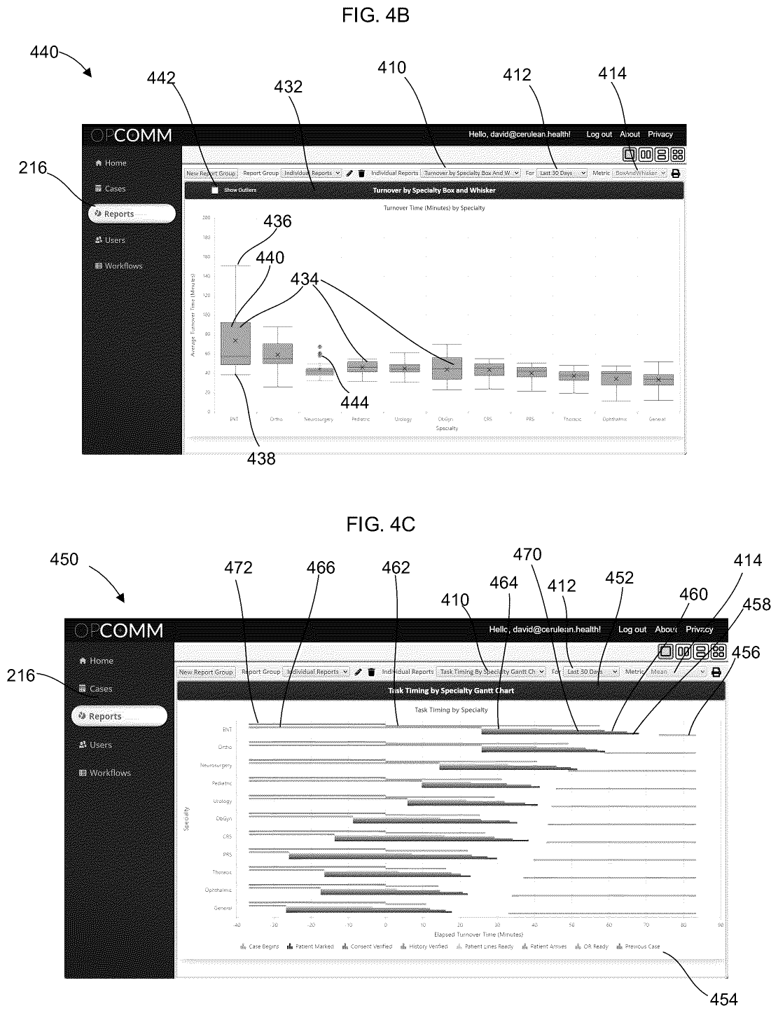

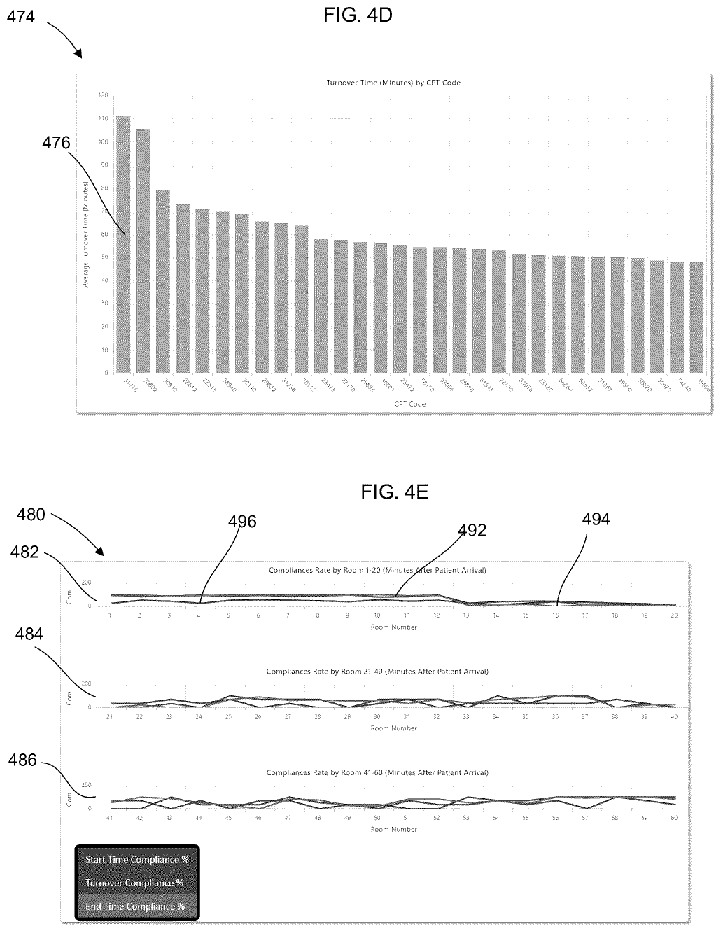

FIG. 4A shows a screen shot of an interface 400 that is displayed when the reports option 216 is selected from the main menu file 210. The reports may be generated based on the collected data from the task lists including the time each task was completed that are stored in the SQL database 130 in FIG. 1. The interface 400 includes a new report group option 402, a report group option 404, and an individual reports option 406. The new report group option 402 allows the user to assemble a custom set of reports that are of special interest to the user, and the reports in such a group may be selected from the group of default reports or custom-designed by the user. Each report group created by a user can be given a name by the user for easier identification after creating multiple report groups. The report group option 404 permits the user to quickly and more conveniently display the reports placed in the custom group. The individual reports option 406 allows the user to select any report that is provided by default to the user.

The reports may be generated according to a set of filters selected by the user and applied by the analytics package 164. Any of the system reports can be used to create a subset of cases for which another report can be used to analyze that subset, and this process may be repeated multiple times. A report-type pull-down menu 410 includes options to select one type of report offered by the analytics package 164. In this example, the report types available include recurring incident case analysis, incidents by room, a summary of cases, case demographics, a turnover time summary, turnover time by specialty, turnover time by hour of the day, turnover time by day of the week, turnover by CPT code, turnover by room, first case (of the day) turnover time compliance by OR room, first case (of the day) turnover time compliance by surgical specialty, task timing by turnover compliance, task timing by start time compliance, task timing by end time compliance, task delay after patient arrival by turnover compliance, task delay after patient arrival by start time compliance, task delay after patient arrival by end time compliance, task delay after patient arrival by operating room, and task timing by specialty Gantt chart. Referring to all reports listed above, the system 100 receives data from the EMR 120, preferably as HL7, and commonly as SCH_16 data, indicating the start times for each surgery, the type of surgery, the room assigned to the surgery, and the attending surgeon assigned to the surgery, and preferably the end times for each surgery, and all surgical team members assigned to the surgery, and optionally maintains a detailed, de-identified, or anonymized historical record of all surgeries, including the procedure type, patient name if applicable, start and end times, patient length of stay, entry and exit times into the post-acute care unit (PACU). When data is stored in an anonymized manner, information that could be used to decode the records to identify a particular person or small group of people is replaced by a system generated code. In contrast, de-identified data is cleansed of information identifying a person but can be re-identified by comparing the information in the historical record to another record or series of records, preferably stored outside the system 100 when it is useful and ethical to re-identify individuals or a group of individuals. Detailed historical records may optionally delete some individual identifying information but can be attributed to an actual individual with no effort or minimal effort. The system 100 also collects data on users' elections to join or leave the surgery. There are two ways to join a case, in one preferred embodiment a user can click a button to join a case, and in another preferred embodiment the system will automatically add a user to the case upon viewing the case and in another preferred embodiment a user is joined to a case if the user takes any action, clicks any button, or otherwise interacts with the case record, and these preferred embodiments may be combined. In one embodiment, the user can leave a case by clicking a button displayed within the case, and in another embodiment, a user can leave a case by swiping across their name or personal identifier on a touch sensitive screen (e.g., to the left, up, down, diagonally or to the right) and these two preferred embodiments optionally may be combined. Preferably, a historical record of surgeries is stored in the system 100. The historical record may be compiled and/or maintained without any personal health information (PHI) to minimize the risk of disclosure of personal and confidential information to people outside of the system. In one embodiment, the historical surgical records and the record of case events for each surgery is stored in separate tables in the same database. In one embodiment, the case history is stored as de-normalized data, which has one advantage of providing for fast database queries. The system 100 calculates the time between activities based on the time stamps and stores the data in a database. Users can then retrieve the data via query and compare to either user-determined benchmarks or benchmarks determined by the system 100 with prior user data from previous time periods.

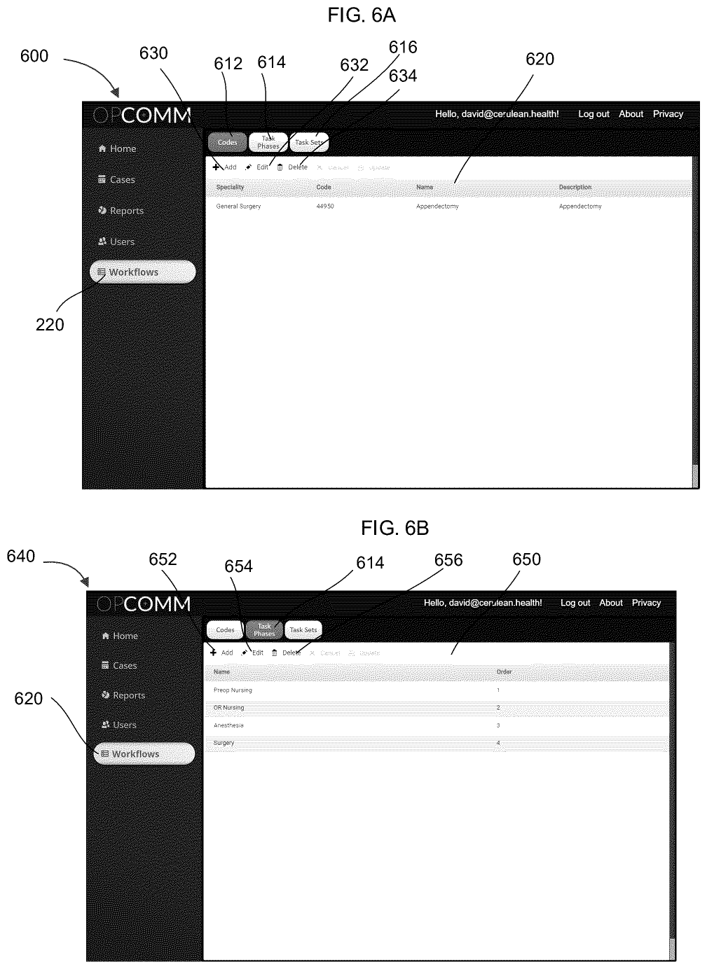

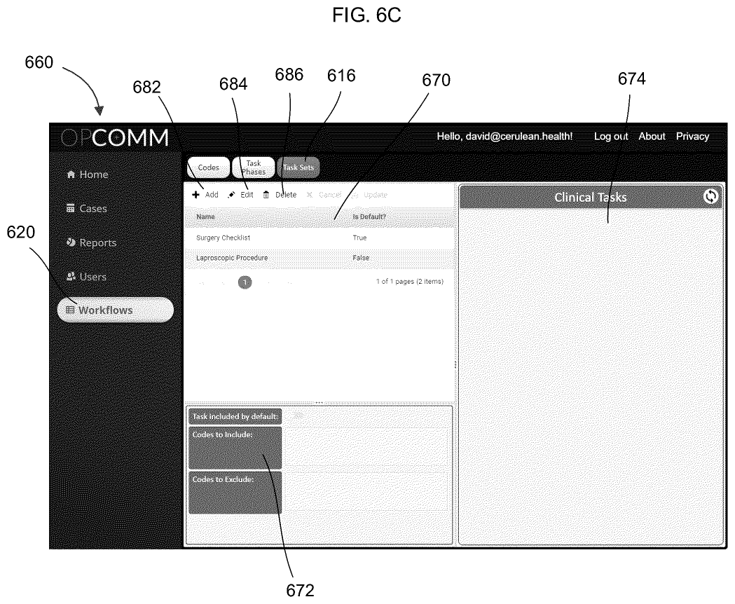

The system 100 provides a collection of reports. One report type is the recurring incident case analysis. The recurring incident case analysis presents the users with a list of cases that deviate from user standards that are either selected manually by the user, or the system calculates based on prior user data. Standards include, but are not limited to, turnover time between surgeries, the time between patient arrival and patient consent, the time between patient arrival and the OR room is ready to receive the patient, the time between patient arrival and placement of intravenous line, the time between patient arrival and confirming the patient's history and physical, time between patient arrival and confirming laboratory studies, the time between patient consent being confirmed and the scheduled surgical start time, the time between the OR room being ready to receive the patient and the scheduled surgical start time, the time between the placement of the intravenous line and the scheduled surgical start time, the time between the confirmation of the patient's history and physical and the scheduled surgical start time, and the time between confirmation of laboratory studies and the scheduled surgical start time. In one embodiment, data points that exceed user or system-assigned benchmarks are classified as incidents. In another embodiment, the system 100 calculates the standard deviation from the mean or median and identifies cases which exceed one, two, or three standard deviations, which are then deemed and/or marked as “incidents” for the recurring incident case analysis report. In another embodiment, the system calculates the top 1 percent, top 2 percent, top 3 percent, top 4 percent, top 5 percent, top 10 percent, top 25 percent, top 50 percent, top 1, top 2, top 3, top 4, top 5, or the top 10 cases and then deems and/or marks them as incidents, wherein the top case is the case in which the Standard exceeds the benchmark by the largest amount of time or extent. In referring to the exceptional cases deemed or marked as incidents, the top x percent and standard deviations optionally can be selected by attending surgeon, CPT code, OR, surgical specialty, surgical pod, other surgical teams members, time slot and/or time of day including specific hours and pre-lunch or post-lunch, day of the week, day of the month, and scheduled case length. This enables an administrative user of the system 100 to identify circumstances common to a particular surgeon, CPT code, OR surgical specialty, surgical pod, other surgical team members, time slot and/or time of day including specific hours and pre-lunch or post-lunch, day of the week, day of the month, and scheduled case length. Upon doing so, the administrative user is enabled to suggest or try workflow and/or policy adjustments to help the surgical teams complete their collective pre-operative tasks in a more efficient manner, and also improves the case status information available to users (surgical team members) to improve their work experience and permit them to better schedule the time in their day left to their personal discretion. For example, a team member may be inclined to go to another part of the hospital but upon checking the system may determine that a better choice, and a choice that will not delay starting a case (i.e., surgery), is to remain near the OR. In one embodiment, the user can select the minimum number of incidents of an incident type to be classified as a recurring incident. When the minimum number of incidents is set to then number 1, then all incidents are displayed in the recurring incident case analysis report. In one preferred embodiment, the system automatically suggests or determines the minimum number of incidents of an incident type to be classified as a recurring incident based on prior observed data. The system can determine the classification of recurring incidents utilizing artificial intelligence, and or machine learning, including but not limited to random forest, k-nearest neighbor, bootstrapping, structured kNN, LASSO, a probabilistic classifier such as Naïve Bayes Classifier, support vector machine (SVM), neural networks, deep learning, large language models, and any other suitable or useful system, methodology, or approach, either individually or in combination. The total recurring incidents are then presented to the user based on queries in a data table. For example, a hospital administrator may want to determine if there are delays occurring between two particular steps in the pre-operative process and manually assign a benchmark that an incident is if the time between the two steps is longer than 3 standard deviations than the median time observed. The system 100 will then retrieve the datapoints according to the user selected preferences of times observed greater than 3 standard deviations above the median time and then presents the datapoints in a data table as an incident report. A range of times for the report may be selected via a time range pull down menu 412. The times may include the last 1 day, last week, last two weeks, last 30 days, six weeks, 60 days, 90 days, or last year. Thus, a range of times may be selected from predetermined options. Alternatively, the user may designate a custom period of time for the time range in the pull down menu 412. A metric may be selected via metric pull down menu 414 (shown in FIG. 6B) that may include displaying the mean, the median, scatter plots, box plots, line charts, scatter plots of outlier datapoints overlaid on a box plot, or scatter plots overlaid on a box plot. Box plots can be configured in a conventional manner, but in one embodiment the box marks the Interquartile Range, a line separating the box into two halves shows the median, an “x” or similar marking shows the mean, and the whiskers shown extend to one standard deviation above and below the mean, and data points outside the whiskers are deemed to be outliers.

In this example, a recurring incident analysis window 420 is displayed from selection of the recurring incident cases option in the pull down menu 410. The window 420 displays a list of recurring incidents 422 and all incident cases 424 within the date range selected through the date range menu 412. The list of recurring incidents 422 lists two different key metrics, the number of cases, the bottleneck turnover, the average turnover and the time lost. The key metrics in this example are the type of surgery (by CPT code) and the task. Other key metrics may be used. The user can select a range of items from the drop down menus based on the metrics they wish to select. A recurring threshold selection menu 426 allows a user to select how many times an event must occur to be considered recurring. In this example, the threshold is selected to be 3 events occurring to be considered recurring.

Another report is the task timing Gantt chart. The task timing Gantt chart presents users with a graph that plots the elapsed time of events, the order in which events occurred, and the workflow dependencies between events. Events presented and/or graphed in the task timing Gantt chart include at least one and optionally all of the following: time between surgeries, the time between patient arrival and patient consent, the time between patient arrival and the OR room is ready to receive the patient, the time between patient arrival and placement of intravenous line, the time between patient arrival and confirming the patient's history and physical, time between patient arrival and confirming laboratory studies, the time between patient consent being confirmed and the scheduled surgical start time, the time between the OR room being ready to receive the patient and the scheduled surgical start time, the time between the placement of the intravenous line and the scheduled surgical start time, the time between the confirmation of the patient's history and physical and the scheduled surgical start time, the time between confirmation of laboratory studies and the scheduled surgical start time and other pre-operative workflow metrics. In one embodiment, users can present Gantt charts across multiple cross-sections, filtered by attending surgeon, CPT code, OR, surgical specialty, surgical pod, other surgical teams members, time slot and/or time of day including specific hours and pre-lunch or post-lunch, day of the week, day of the month, and scheduled case length. In one embodiment, events that exceed user or system-assigned benchmarks are classified as incidents. In one embodiment, the user can select the minimum number of incidents of an incident type to be classified as a bottleneck. In another embodiment, the system calculates the standard deviation from the mean or median and identifies events which exceed one, two, or three standard deviations which are then deemed and/or marked as “bottlenecks” for the task timing Gantt chart. In another embodiment, the system calculates the top 1 percent, top 2 percent, top 3 percent, top 4 percent, top 5 percent, top 10 percent, top 25 percent, top 50 percent, top 1, top 2, top 3, top 4, top 5, or the top 10 events and then deems and/or marks them as bottlenecks, wherein the top events exceed the benchmark by the largest amount of time or extent.

The list of all incident cases 424 lists the case number, the specialty, the CPT code, the hour of the day, the OR location, the person, the task, and the turnover time. A deviation selection menu 428 allows a user to select how many standard deviations from the mean or median the case event must occur before it is considered an event. In this example, the cases outside of one standard deviation of the mean are displayed. This information is useful to OR administrators and surgical teams because it identifies a collection of cases that are exceptional in their failure to meet turnover time, first case start on time (FCOS), or last case on time end (LCOTE), or two of the preceding, or all three. The user or administrator can then use other reports within the system, such as the Gantt chart, to identify which (if any) of the Case Events tracked by the system are anomalous. In one hypothetical, the incident report indicates that 9 surgeries out of 50 of identified by the same CPT code had exceptionally high turnover time. In this hypothetical, it occurs to the team that two types intraoperative imaging devices can be in surgeries with the hypothetical CPT code. Another insight may be that 8 of the 9 surgeries use the second type of imaging equipment. Further, only 11 surgeries using the second type occurred in the period of time that was analyzed. The OR administrator inquires and finds out the second type of imaging equipment is not kept in the same branch of the OR suite and belongs to a surgical pod other than the one the performs the hypothetical surgeries identified by the hypothetical CPT code. By consulting the Gantt chart generated by the system 100, the hypothetical team discovers that all case events are completed more than 40 minutes prior to the beginning of the exceptionally-delayed surgeries except for the case event indicating that the patient can be transported from pre-anesthesia to the OR. One advantage of the system 100 is that by tracking the timing of case events, novel data types are generated that facilitate system identifying the cause of bottlenecks and delays in the pre-surgical workflow. Given the high cost of every minute of OR time, the hospital achieves a high return on its investment of time and resources using the system 100.

FIG. 4B shows an interface 430 that is displayed when the turnover by specialty option is selected in the pulldown menu 410. A turnover by specialty graph 432 is shown that is filtered by the date range selected in the date range menu 412 and the metric menu 414. The turnover by specialty graph 432 shows a set of bars 434 for each specialty such as orthopedics, neurosurgery, or general surgery. Each of the bars 434 includes a top whisker 436 and a bottom whisker 438 and a box area 440. The box area 440 represents the inter-quartile range (i.e., the middle 50% of cases, “IQR”). The whiskers 436 and 438 show the range capped at 2 standard deviations from the mean. An “X” shows the mean and a bar shows the median within the box area 440.

A radio box 442 may be checked to show remaining data points 444 that may be shown as outliers as a dot plot on the bars 434. Selecting any of the box areas 440 results in a bar graph that displays surgeries in that specialty shown in groups according to CPT codes.

FIG. 4C shows an interface 450 that is displayed when the task timing by specialty report is selected in the pulldown menu 410. A task timing by specialty Gantt graph 452 is shown that is filtered by the time range selected in the date range menu 412 and the metric menu 414. Each specialty is listed in the vertical axis. The horizontal axis represents time units before and after the beginning of the procedure and in this example the end of the prior case is shown at the zero on the time axis.

A key field 454 shows different icons representing different information given by the lines corresponding to each of the specialties. Different colored icons may be selected from the key field 454 to display different lines in the graph 452 representing case events. The case events include patient arrival (when the patient arrives in the area under control by the OR teams), OR ready time (when is the operating room clean and set up for the next case including mopping (if required), new linens on the bed, and the appropriate tools present in the room), case beginning (which can be set to the time the patient's transport bed is rolled into the OR), consent verified (meaning the consent for the procedure is on record in the EMR and correct for the procedure; additional consents (if needed) can be listed here too), history verified (meaning that the patient's medical record has been reviewed and no contraindications to the procedure are present), patient lines ready (meaning the tubing and insertions into the patient are present so that medications and fluids can be delivered to the patient per the standard operating procedure), patient marked (usually performed by the surgeon, indicating that the left and right body parts have been indicated with a physical marking on the patient to specify either where surgery is required, where it should not be performed or both), and case begins. Any of the case events (specific tasks) listed in the field 454 can be selected and these will be plotted for the parameters selected in the header for ease of analysis. This helps the administrative staff understand where possible bottlenecks appear. Moreover, the time axis can be set to zero when the prior case ends, in which case some events may occur in negative time space but most will have occurred in positive time space; or in an alternative embodiment, another time point such as “case begins” may be selected for the zero time point and the chart correctly displays lines of a length to indicate the time differences between the time axis-origin and the time at which the event has occurred.

If the respective icon in the key field 454 is not selected, then the corresponding line is not shown on the graph, which has the effect of removing visual information or visual clutter from the graph 452. Thus, in this example, all of the icons representing different events have been selected and thus a turquoise line 456 represents the case beginning, a black line 458 represents the patient being marked, a dark blue line 460 represents consent verified, a red line 462 represents history verified, an orange line 466 represents that lines are ready, a green line 468 represents when a patient arrives, a pink line 470 represents the time the OR is ready, and a light blue line 472 represents the end time of a previous case.

FIG. 4D shows an interface 474 that is displayed when the turnover time by CPT code option is selected in the pulldown menu 410. In this example, the turnover time is represented on the y-axis and the bars of the bar graph are labeled with the applicable CPT code. The interface 474 shows a graph 476 with each CPT code on the horizontal axis. Each bar represents the turnover time for the procedure represented by the CPT code based on the scale on the vertical axis. Procedures with two CPT codes are displayed twice, one time within each CPT code, however if the case record is marked with a primary CPT code and one or more secondary CPT code(s), then the turnover time for such a case would only be represented in the report for the primary CPT code. The bars on the graph 474 are derived from data that is filtered by the time range selected in the date range menu 412 and the metric menu 414.

FIG. 4E shows an interface 480 that is displayed when the compliances by room report is selected in the pulldown menu 410. A case is deemed to be compliant when: (a) the first case starts on time; (b) has a turnover time less than or equal to the goals (measured in minutes) for turnover time set by the facility's management team; and/or (c) when the last case of the day ends before the end of the scheduled day. The compliance report represented by the interface 480 shows the percentage of cases that meet each of these three forms of compliance. While this example shows the percentage compliance by room, the same analysis can be done according to a particular type of classifier such as the assigned surgeon, the surgical pod, the surgical specialty, each CPT code, or collections of individual CPT codes. Any number of rooms (or other classifiers) can be collected together in a single graph but for convenience of observation and uniformity, in this example 20 rooms are displayed on each graph. A set of compliance graphs 482, 484, and 486 that are filtered by the time range selected in the date range menu 412 and the metric menu 414. In this example, each of the compliance graphs represent time ranges after patient arrival. Each of the graphs 482, 484, and 486 plot lines representing each compliance percentage average of all the operating rooms at the different times on the horizontal axis. Thus a line 492 represents the compliance percentage of start times. A line 494 represents compliance percentage of end times. A line 496 represents compliance percentage of turnover time. Each of the lines 492, 494, and 496 may be rendered in a different color to distinguish the different compliance percentages.

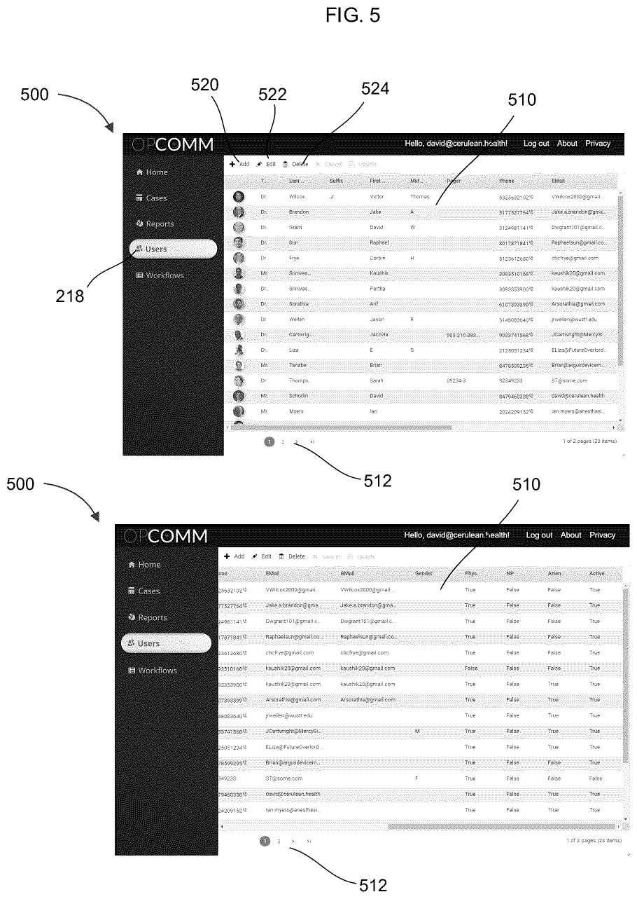

FIG. 5 shows an interface 500 shown when the users option 218 is selected from the main menu 210. The interface 500 allows a user to be added to the system 100 with contact information, along with roles and other information. The interface 500 includes a current listing window 510 that lists all current users. A bottom navigation bar 512 allows different pages of listings to be displayed. Each user entry includes a photo icon, name information, pager information, phone number, email, alternate email, gender, whether the user is a physician, whether the user is a nurse, whether the user is an attending physician, and whether the user is active. An add button 520 allows an administrator to add a user. The add button 520 causes different pop up windows to appear to allow the entry of the data fields listed above. Such fields may be partially or fully populated by a new user and an administrator may only need to approve the entry of the new user and associated information. An edit button 522 allows an administrator to edit the information for a user in any of the fields listed. A delete button 524 allows an administrator to delete a user.

The system 100 allows an administrator to edit codes, task phases and task sets for the tasks listed by the interfaces for each of the team members by selecting the workflows option 220. The system 100 thus may be used to create customized workflows through the administration module 148 in FIG. 1. For example, an administrator may add a button that asks for a supplementary informed consent from a patient, when the administrator believes that the current signed informed consent could later be deemed to be invalid, e.g., when the patient could be cognitively impaired, potentially is not of age, or when applicable laws require a second type of informed consent. In a second example, an administrator may be aware that surgeries that are identified by, or associated with, a particular CPT code require the surgical staff to locate some specialized apparatus or piece of equipment that may be available in the surgical suite but not routinely located in a specified location. Because the surgery cannot start in this example until the equipment is located, clean and in the OR, other members of the surgical team would need to know when these conditions are met in order to be efficient in their use of time. Accordingly, in this second example the administrator may add a task such as “augmented reality visor is in the OR” to the list of case tasks. The system is then able to track whether there is a correlation between delays in clicking the “augmented reality visor is in the OR” button and long turnover times.

Workflows of team members are monitored by the system 100 through data indicating the time of completion of tasks that are correlated to the procedure, team, and operating room. The workflow may be customized through creating and editing lists of case events (i.e., tasks) to be checked-off by team members using the mobile application 170. FIG. 6A shows an interface 600 that is displayed when the workflows option 220 is selected. The interface 600 includes a CPT code option 612, a task phases option 614, and a task sets option 616. In this example, the CPT code option 612 has been selected. The interface 600 thus allows a task to be added or subtracted from the tasks to be completed for each CPT code. The tasks are added to the system 100 with associated procedures and other information. The interface 600 includes a current code listing window 620 that lists all current codes. Each code entry includes the associated specialty, name, and description. An add button 630 allows an administrator to add a code. The add button 630 causes different pop up windows to appear to allow the entry of the new code, associated specialty, name and description. An edit button 632 allows an administrator to edit the information for an existing code selected from the code listing window 620. A delete button 634 allows an administrator to delete a user that is selected from the code listing window 620. While the codes are CPT codes in this example, any codification systems, such as ICD-10 codes (or their predecessors or successor ICD codes) may be used.

FIG. 6B shows an interface 640 that is displayed when the task phases option 614 is selected. The interface 640 allows a task phase to be added to the system 100. The task phase generally is associated with a type of team member such as the Pre-op nurse, OR nurse, surgeon, and anesthesiologist. The type of team member may be given a different name, and thus it is to be understood that the Pre-op nurse, OR nurse, surgeon, and anesthesiologist are examples. Team members with similar responsibilities may be given different names such as a circulating nurse instead of the “OR nurse.” The interface 640 includes a current task phase listing window 650 that lists all current task phases. Each task phase entry includes a description and an order. In this example, the task templates are designated as a default to all templates or unique to specific CPT codes. An add button 652 allows an administrator to add a task phase to the listing window 650. The add button 652 causes different pop up windows to appear to allow the entry of the new task phase and the relative order in relation to existing task phases. An edit button 654 allows an administrator to edit the information for a selected task phase in the listing window 650. A delete button 656 allows an administrator to delete a selected task phase in the listing window 650. The bottom bar includes page navigation icons that allow a user to navigate through multiple pages of templates.

FIG. 6C shows an interface 660 that is displayed when the task sets option 616 is selected. The interface 660 allows tasks to be added to the system 100 with associated procedures and other information. The interface 660 includes a current task listing window 670 that lists all current tasks. Each task entry includes the name of the task and a Boolean indicator whether the task is a default task. A procedure code window 672 lists whether the task entry is a default task across all templates or is a unique task for a set of procedures based on including or excluding CPT codes. A clinical task window 674 lists the task entry items for a selected procedure template once selected. An add button 680 allows an administrator to add a task entry. The add button 680 causes different pop up windows to appear to allow the entry of the new task, associated specialty, name, and description. An edit button 682 allows an administrator to edit the information for an existing task in the task listing window 670. A delete button 684 allows an administrator to delete a selected task from the task listing window 670.

The above described system 100 provides a mechanism for reducing turnover time by real-time coordination by a surgical team being updated by completion of pre-operation tasks by each team member. In a hypothetical example, a surgeon needs to leave the OR Suite and travel to a medical unit for in-patients to check on patients from the prior days' surgery. In this hypothetical, however, the surgeon checks the system 100 prior to leaving the surgeon's desk, and realizes that the next surgery is about to begin. The surgeon instead uses the next five minutes completing paperwork at the surgeon's desk. When the system 100 alerts the full team that all pre-surgical tasks have been completed such that the patient may be transported from pre-anesthesia to the assigned OR, the surgeon is still within the OR Suite instead of on the medical care floor speaking with a patient and one of the patient's family members. Having active and interactive information about the progress of other team members reduces OR turnover time. Because this hypothetical case is not delayed, the last case of the day scheduled in the same OR can proceed instead of being postponed due to a series of extended turnover times in the schedule ahead of it. Alternatively, the team is able to finish the last case of the day on time avoiding costly overtime pay and frustrating the team members who have commitments after the work day is scheduled to end. The system 100 includes a module to transmit real-time information about the completion of tasks and cases to the server 120 for automatic adjustment of scheduling of teams in a scheduling application run by the server 120. For example, the nurse anesthetist meets a patient in the pre-anesthesia area, and has the patient sign an informed consent document authorizing the use of anesthesia during the impending operation, to the nurse anesthetist marks a the case event indicator corresponding to receiving the consent for anesthesia. However, the circulator nurse tells the nurse anesthetist that the surgeon has notified the circulator nurse in the hallway that the surgeon is being called out of the OR Suite for an emergency. Accordingly, the nurse anesthetist does not start the patients intravenous lines and instead leaves the area to attend to other matters. After attending to the emergency, the surgeon has to decide whether to stay on the medical care floor to attend to other matters as long as the surgeon is already on the medical floor. Fortunately however, the surgeon checks the system 100 and sees that all other case events except for starting the intravenous lines and the surgeon's own pre-surgical case events. This means that turnover time is now being extended and the surgeon directly returns to the OR suite to keep the surgeries starting at their scheduled times. Before heading back, the surgeon sends a text message to the team stating, “heading back, scrubbing in in 5”. The anesthetist sees the message and promptly starts the intravenous lines. The communication enabled by the system 100 enables better team decision making and coordination of activities.

The real-time information relating to completion of tasks (i.e., case events) may also be transmitted to physical indicators such as display screens or colored lights around an operating room to indicate the status of the procedures being performed.

Further, geolocation data may be sent from the user devices to displays in proximity of an operating room to indicate the presence of team members, the type of procedure, and other useful operational information.

The system 100 allows a hospital or other health care facility to reduce turnover time. Excess turnover time prevents operating rooms from hosting as many surgeries as possible in a working day thus facilitating treatment of patients. Excess turnover time also causes hospitals to incur overtime costs for nursing and anesthesiology personnel. Finally, excess turnover time also decreases workplace morale and satisfaction. The system may be applied to other health care environments such as dentistry, emergency room care, intensive care units (ICU)s, and Post Anesthesia Care Units (PACU).

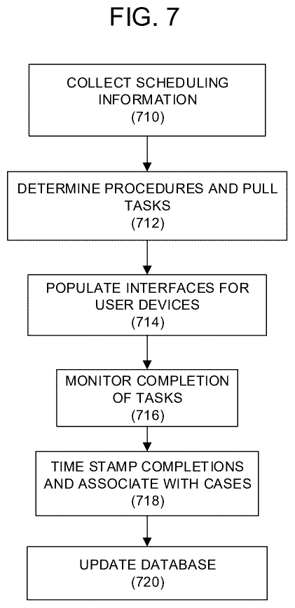

The operation of the example system 100 shown in FIG. 1, which may be controlled on the example server and user devices, will now be described with reference to FIG. 1 in conjunction with the flow diagram shown in FIG. 7. The flow diagram in FIG. 7 is representative of example machine readable instructions for implementing the application to collect and manage task list data from team members performing health care procedures. In this example, the machine readable instructions comprise an algorithm for execution by: (a) a processor, (b) a controller, and/or (c) one or more other suitable processing device(s). The algorithm may be embodied in software stored on tangible media such as, for example, a flash memory, a CD-ROM, a floppy disk, a hard drive, a digital video (versatile) disk (DVD), or other memory devices, but persons of ordinary skill in the art will readily appreciate that the entire algorithm and/or parts thereof could alternatively be executed by a device other than a processor and/or embodied in firmware or dedicated hardware in a well-known manner (e.g., it may be implemented by an application specific integrated circuit (ASIC), a programmable logic device (PLD), a field programmable logic device (FPLD), a field programmable gate array (FPGA), discrete logic, etc.). For example, any or all of the components of the interfaces could be implemented by software, hardware, and/or firmware. Also, some or all of the machine readable instructions represented by the flowchart of FIG. 7 may be implemented manually. Further, although the example algorithm is described with reference to the flowchart illustrated in FIG. 7, persons of ordinary skill in the art will readily appreciate that many other methods of implementing the example machine readable instructions may alternatively be used. For example, the order of execution of the blocks may be changed, and/or some of the blocks described may be changed, eliminated, or combined.

FIG. 7 is a flow diagram for the process of executing the example system 100. The routine first collects scheduling information regarding cases and assigned team members from the EMR server 120 (710). The routine then determines the types of procedures for each case and pulls associated task list data (712). The routine then populates the task list interfaces for all team members associate with each of the scheduled cases (714). The routine then monitors the input of task completion by team members (716). The routine then time stamps all completed tasks and associates the tasks with factors such as the operating room, team member, procedure, case and the like (718). The routine then updates the SQL database 130 (720) for access by the analytics package 146.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A computer implemented method for coordination of tasks for a surgical medical procedure team of medical professionals performing a surgical procedure, the method comprising:
receiving scheduling information for the medical procedure, the scheduling information including a plurality of team members with computer devices assigned to perform the surgical procedure and a set of tasks associated with each of the plurality of team members prior to commencing the surgical procedure, wherein each of set of tasks for each of the plurality of team members is specific for performance by the team member for either preparing or performing the surgical medical procedure;
providing the set of tasks to each of the team members on the computer devices;
generating a task list interface on each of the computer devices that includes an input to indicate completion of tasks on the set of tasks associated with the team member with the computer device;
communicating to the computer devices of all other team members that a task has been completed from any of the team members; and
recording when a task has been completed on all the computer devices of all the team members on the task list interface.

2-48. (canceled)

49. A system for monitoring surgical procedures, the system comprising:
a storage device storing schedule data of surgical procedures, the schedule data including members of a surgical procedure team assigned to perform the surgical procedure and task lists for each member of the surgical procedure team for a surgical procedure, wherein each of set of tasks for each of the plurality of team members is specific for performance by the team member for either preparing or performing the surgical procedure;
a communications interface to communicate with user devices each associated with a member of a team to perform the surgical procedure; and
a controller coupled to the storage device and communication interface operable to:
send a task list to the user device associated with each of the members of the surgical team based on the schedule data on a display;
receive inputs from one of the user devices indicating the completion of a task on the task list; and
send a signal for the user devices associated with the surgical team members updating the task lists to indicate the completion of the task.

50-64. (canceled)

65. A non-transitory, machine readable medium having stored thereon instructions for monitoring preparation for a surgical procedure, the stored instructions comprising machine executable code, which when executed by at least one machine processor, causes the machine processor to:
receive scheduling data for the surgical procedure, the scheduling data including a plurality of team members with computer devices assigned to perform the surgical procedure and a set of tasks associated with each of the plurality of team members prior to commencing the medical procedure, wherein each of set of tasks for each of the plurality of team members is specific for performance by the team member for either preparing or performing the surgical procedure;
provide the set of tasks to each of the team members on the computer devices;
generate a task list interface on each of the computer devices that includes an input to indicate completion of tasks on the set of tasks associated with the team member with the computer device;
communicate to the computer devices of all other team members that a task has been completed from any of the team members; and
record when a task has been completed on all the computer devices of all the team members on the task list interface.

66. The system of claim 49, wherein the server is operable to generate a report based on the recording of the completed tasks and completed tasks for other teams and other surgical procedures.

67. The system of claim 66, wherein the report includes one of recurring incident cases, turnover time by specialty, task timing by specialty, turnover time by code, and compliance by room.

68. The system of claim 66, wherein the report is limited by filters of the completed tasks.

69. The system of claim 68, wherein the filters include time period, surgical room, specialty, medical procedure code, or disease code.

70. The system of claim 69, wherein the medical procedure code is a Current Procedural Technology (CPT) code, and wherein the disease code is an International Classification of Diseases Revision 10 (ICD-10) code.

71. The system of claim 49, wherein the set of tasks are standard across a plurality of different types of surgical procedures, the plurality of surgical procedures including the surgical procedure.

72. The system of claim 49, wherein each task list is specialized according to at least one of the group consisting of: (a) an identity of a first physician; (b) an identity of a first and a second physician; (c) a medical procedure identifier code; (d) a disease or condition code; or (e) a location of an operating room.

73. The system of claim 49, wherein the team is extracted from at least one of an electronic health record, an electronic medical record, or an emergency procedure case report generated by emergency medical service providers or wherein the team is created by an individual selecting the case from a list of cases displayed from the scheduling information.

74. The system of claim 49 wherein the server is operable to add a second surgical procedure in real-time to a schedule, wherein the surgical procedure is part of the schedule.

75. The system of claim 49, wherein the task list interface includes at least one dynamic case status indicator.

76. The system of claim 75, wherein the dynamic case status indicator is indicated in a first color when all tasks are completed, a second color when at least one task is incomplete, and a third color when the medical procedure has been canceled, the patient has entered the operating room, or the medical procedure has begun.

77. The system of claim 49, wherein the server allows communication between team members or and an individual associated with the surgical procedure via the computer devices.

78. The system of claim 49, wherein the controller is operable to display an interface showing identification of all team members.

79. The system of claim 49, wherein the surgical procedure is one of a plurality of surgical procedures assigned to one of the team members, and wherein the controller is operable to:
display a list of the plurality of surgical procedures assigned to the team member; and
display a list of tasks of the team member associated with each of the plurality of surgical procedures, when one the plurality of surgical procedures is selected, and wherein each of the plurality of surgical procedures includes a graphic indicator of the status of the tasks of each team member assigned to the procedure.

80. The system of claim 49, wherein the controller displays an interface showing information for a patient's medical representative.

81. The system of claim 49, wherein one of the tasks is completion of a document, wherein an input allows display of the document on each user device.

82. The system of claim 49, wherein the controller is operable to display a multi-case view showing the status of a plurality of surgical procedure teams, wherein the plurality of surgical procedure teams includes the surgical procedure team.
